(19) United States
(12) Patent Application Publication
(10) Pub. No.: US 2025/0186252 A1
(43) Pub. Date: Jun. 12, 2025

(54) METHOD AND SYSTEM FOR DELIVERY OF THERAPEUTICS TO EYE

(71) Applicant: The Johns Hopkins University, Baltimore, MD (US)

(72) Inventors: Peter A. Campochiaro, Baltimore, MD (US); Jordan J. Green, Woodstock, MD (US); Kunal Parikh, Reynoldsburg, OH (US)

(21) Appl. No.: 18/844,364

(22) PCT Filed: Mar. 7, 2023
(86) PCT No.: PCT/US2023/014751

§ 371 Date: Sep. 5, 2024

Related U.S. Application Data
(60) Provisional application No. 63/317,410, filed on Mar. 7, 2022.

(51) Int. Cl. A61F 9/00

(52) U.S. Cl. CPC A61F 9/0017

(57) ABSTRACT

A surgical instrument is provided for delivery of therapeutic agents into an eye. The instrument includes a cannula that is connected to a handle of the instrument and through which therapeutic agents are delivered into the eye via a continuous and controlled infusion. In certain aspects, a sheath which has a diameter greater than that of the cannula accommodates the cannula therein. An insertion tip is formed at a distal end of the cannula to penetrate tissue of the eye at a tangential angle.

### CROSS-REFERENCE TO RELATED APPLICATION

This application claims the benefit of U.S. provisional application 63/317,410 filed Mar. 7, 2022, which is incorporated by reference herein in its entirety.

### GOVERNMENT SUPPORT

This invention was made with government support under grant EY031097 awarded by the National Institutes of Health. The government has certain rights in the invention.

### TECHNICAL FIELD

The present disclosure relates to a surgical instrument and method of operating the same, and more particularly, to a surgical instrument and method of operating the same to deliver therapeutic agents to the eye in a minimally invasively manner.

### BACKGROUND

Generally, today the delivery of therapeutics or genes to the back of the eye includes methods such as subretinal injection, intravitreal injection, microinjection, and catheter-based delivery. Research is actively being conducted on gene therapy and its use for treatment of ocular diseases by repairing defective genes or by insertion of therapeutic genes. The use of gene therapy for ocular procedures is compelling due to the anatomy of the eye restricting systemic exposure of viral or non-viral gene and also the retina generally limits inflammatory response.

FIGS. 1-3 show illustrations of subretinal and intravitreal injections into the retina according to the related art. A subretinal injection is often part of a surgical procedure such as vitrectomy. However, subretinal injections have various disadvantages. In particular, the procedure for a subretinal injection is quite an invasive process and requires administration by surgical procedure which increases both the surgical length of time as well as overall procedure costs. Additionally, vitrectomy procedures require a substantial recovery time and have an increased risk of post-operative complications. Another risk with a subretinal injection is that the injection under the fovea of the eye may cause separation of central photoreceptors from the retinal pigment epithelium. Such a separation may cause damage to the photoreceptors and permanent reduction in visual acuity. The pressure of the subretinal injection under the macula of the eye may cause dehiscence through the thinnest part of the retina or fovea which results in a macular hole.

Although an intravitreal injection of therapeutic agents does not require a surgical procedure like the subretinal injection, intravitreal injections have limited penetration into underlying target layers of the retina due to the inner limiting membrane, as shown in FIG. 1.

FIGS. 2 and 3 illustrate the theoretical penetration of the therapeutic agents using the subretinal and intravitreal injections, respectively. In particular, the figures show the distribution of adeno-associated virus (AAV) vectors that are used to deliver genes to the eye. FIG. 2 illustrates the subretinal injection of therapeutic agents through the retinal pigment epithelium (RPE). As shown in FIG. 3, the AAV transduction through intravitreal injection is limited to areas where the inner limiting membrane is thin, retinal ganglion cells (RGC) around the fovea and adjacent to blood vessels and transitional epithelial cells in the pars plana, thus limiting efficacy.

Furthermore, FIGS. 4A-4B illustrate a microinjector according to the related art. This microinjector allows for a non-surgical procedure for administering therapeutics to the eye and specifically to the suprachoroidal space (SCS) of the eye. The microinjector includes a handle, a barrel showing increments of capacity therein, a needle, and a safety cap. The needle is also surrounded by a compressing hub, as shown in FIG. 4B. This particular variation shows a free length of the needle of 0.9-1.1 mm. FIG. 5 illustrates the use of the microinjector of FIG. 4A for administering the therapeutic agent.

In particular, as shown in FIG. 5, the microneedle is positioned perpendicular to the intended access point of the eye to the penetrate the sclera and deliver the therapeutic agent to the SCS. The arrows in the figure indicate the flow direction of the agent through the SCS region upon infusion. The therapeutic agent then spreads posteriorly and circumferentially. Although this method avoids the need for a surgical procedure and prevents bleb formation while providing distribution of the agent, there are several disadvantages. In particular, the delivery location is limited and controlled by the length of the microneedle which limits reproducibility. Additionally, the payload amount of the therapeutics is limited by the physiological constraints of the SCS. The flow rate of the therapeutics is also uncontrollable. That is, the entire payload is delivered in about 5 seconds and is inconsistent without capability of controlling the rate of fluid introduction. The penetration of the sclera from a perpendicular angle reduces the efficiency of spatial coverage of the retina.

Accordingly, delivery of therapeutic agents to the retina remains a challenging procedure and there is a need for providing an improved surgical instrument capable of providing controlled delivery of therapeutic agents via minimally invasive ocular procedures.

### SUMMARY

In one aspect, we now provide new methods for performing minimally invasive intraocular procedures that do not require a surgical operation.

More particularly, the present methods and systems include tangentially administering a therapeutic agent to the suprachoroidal space (SCS) of the eye to reduce intraocular pressure caused during conventional perpendicular injections of therapeutic agents. The therapeutic agents or gene carriers can be delivered through a controlled infusion to provide optimal delivery of maximum volume of agents.

We also provide surgical instruments for delivery of therapeutic agents to the eye and a method of operating the surgical instrument for a minimally invasive procedure to deliver the therapeutic agents in a continuous and controlled manner.

According to an aspect of the present disclosure, a method of treating an eye of a subject includes tangentially administering a therapeutic agent to the eye to infuse the therapeutic agent to a suprachoroidal space (SCS) of the eye. The therapeutic agent may be administered to the eye at an entry of the eye that is at varied degrees from a perpendicular center line of the eye. The varied degrees may be 5, 20, or 30 degrees as an example. In particular, the eye may be penetrated and the therapeutic agent is administrated through the penetration site.

Additionally, the tangential administration of a composition that comprises the therapeutic agent in a given time period results in pressure within the SCS that is reduced compared to the pressure within the SCS that results by perpendicular administration of the same volume of the therapeutic agent composition within the same time period. The tangential administration results in SCS pressure that is 5, 10, or 20 percent less relative to the perpendicular administration.

Despite the advantages of tangential administration into the SCS, it has been deemed as not viable due to the difficulty of controlling insertion depth and angle, which may increase the likelihood of inadvertent intravitreal or subretinal injections. The present invention, including both methods and instruments, overcome these existing challenges to enable safe, effective, and reproducible administration of therapeutics tangentially into the SCS.

In one embodiment, the method may further include penetrating the eye at a tangential angle with an insertion tip formed at a distal end of a cannula through which the therapeutic agents are delivered into the eye via a continuous and controlled infusion. A sheath is then deployed, wherein the sheath has a diameter greater than that of the cannula and in which the cannula is housed. The therapeutic agents are then directly delivered via infusion tubing connected to the cannula. In this embodiment, the insertion tip is a needle.

In another embodiment, the method may further include penetrating the eye at a tangential angle with an insertion tip formed at a distal end of a cannula through which the therapeutic agents are delivered into the eye via a continuous and controlled infusion. In certain aspects, a sheath having a diameter greater than that of the cannula is deployed and the cannula is housed in the sheath. In certain other aspects, a device does not include such a sheath element. The cannula is then released into the eye. Thereafter, infusion tubing may be connected to the cannula to deliver the therapeutic agents therethrough. The infusion tubing is suitably connected to a controlled flow system to provide controlled flow rates of the therapeutic agents. In this embodiment, the insertion tip is a closed end of the cannula and the eye is penetrated first by sclerotomy.

According to one aspect, a surgical instrument for delivery of therapeutic agents into the eye is provided. In particular, the surgical instrument suitably includes a cannula that is connected to a handle of the surgical instrument and through which therapeutic agents are delivered into the eye via a continuous and controlled infusion. In certain aspects, the instrument suitably further includes a sheath having a diameter greater than that of the cannula and in which the cannula is housed. In other aspects, the instrument may not include such a sheath. An insertion tip is suitably formed at a distal end of the cannula to penetrate tissue of the eye at a tangential angle and particularly, the suprachoroidal space (SCS) of the eye. The agents may also be delivered into multiple quadrants of the eye. The sheath then encloses the insertion tip. The tangential angle of the insertion tip suitably may be about 0-15 degrees, or more, such as 3-45 degrees or 5-45 degrees.

In certain aspects, the surgical instrument suitably comprises a needle element that is preferably 10 cm or less in length, more typically about up to or less than 8, 7, 6, 5, 4, 3 or 2 cam in length.

In certain preferred aspects, the needle tip has a solid end portion that is advanced into a patient's eye. The solid portion may contain a material of construction (e.g. a stainless steel) through the tip cross-section without any type of internal lumen. A solid tip portion suitably also may not include an external openings or ports.

According to an exemplary embodiment, the instrument may further include a manipulator that is provided at the handle. In this embodiment, the manipulator may deploy the sheath (where the device includes a sheath) once the tissue has been penetrated. The sheath may include an overcap that connects to the tissues as the sheath is deployed. The cannula may be a trocar cannula or an infusion cannula. The instrument may also include at least one sensor at a tip of the cannula to output a signal that indicates penetration into the tissue of the eye and an angle change of the cannula with respect to the eye. The cannula may have a length of about 1 to 5 mm. Additionally, the cannula may include one or more openings through which the therapeutic agents flow. The insertion tip may be about a 25-30 gauge needle. Alternatively, the insertion tip may be a closed end of the cannula. The cannula may include flanges to hold the cannula in place against the eye.

According to one exemplary embodiment, the surgical instrument may include infusion tubing attached to the cannula via the handle. The infusion tubing is connected to a controlled flow system to provide controlled flow rates of the therapeutic agents. The cannula may also be bent at a particular angle to position the cannula on the eye. According to another exemplary embodiment, after penetration of the insertion tip into the tissue, a trocar cannula may remain indwelling. Thereafter, infusion tubing is connected to the trocar cannula to deliver the therapeutic agents.

According to one exemplary embodiment, the surgical instrument may include a needle comprising a solid tip. Thus, the solid tip portion does not contain any apertures or openings. A solid tip portion will contain a material of construction (e.g. a stainless steel) throughout the tip cross-section. It has been found that a solid tip portion can effectively ocular tissue.

The needle tip portion is preferred connected to a hollow pencil point cannula in order to provide facile insertion into the SCS via the trocar blade and delivery of the therapeutic agent from an outlet positioned on the length of the cannula shaft inserted into the SCS. The needle may be positioned such that the outlet is directed towards the sclera in order to minimize pressure on the vitreous body and risk or perforation.

In certain preferred aspects, the overall needle size may range from 25-30 gauge with between 1-5 mm of the needle inserted into the SCS. In certain aspects, up to or less than 1, 2, 3 or 4 mm of the needle inserted into the SCS during use. The needle suitably may be connected to a syringe for direct injection into the suprachoroidal space or to tubing to allow for infusion via a pump.

In certain systems, a marker is present on the needle to indicate the optimal length of needle inserted into the SCS. This can be suitably provided by one or more visual marker or a physical or tactile marker or ridge achieved via laser or mechanical methods. In certain systems, a physical stopper is present on the needle to ensure optimal length of needle insertion into the SCS. This may be achieved via a change in diameter of the needle or additional material around the needle (e.g., sheath or cannula).

In certain preferred aspects, a surgical instrument is provided such as for injection or infusion administration to a subject, the surgical instrument comprising: (a) a needle element having (i) an insertion length of 5 mm or less; and (ii) a tapered insertion point. In certain aspects, the needle element has an insertion length of at least 0.5 mm. In certain aspects, the needle element has an insertion length of at least 1 mm. In certain aspects, the needle element insertion point (or tip portion) is a solid element through the cross-sectional width.

In certain aspects, preferred needle devices include a needle element having an outer diameter of 520 μm to 310 μm.

In certain aspects, preferred needle devices include a needle element having an inner diameter of 150 μm to 270 μm. In certain aspects, the needle element has an inner diameter of less than 150 μm, or less than 100 μm.

In certain aspects, preferred needle devices include a needle element that is 25-30 gauge, including 25, 26, 27, 28, 29 or 30 gauge.

In certain aspects, preferred needle devices include a needle element that comprise one or more orifices. Such orifices can permit administration of a composition (such as a fluid composition) from and out of a needle element to tissue and the patient. In certain aspects, the one or more orifices do not circumscribe the needle element, for example a portion of the needle shaft (e.g. 10, 20, 30, 40, 50, 60, 70 or 80 degrees or more of the shaft do not contain orifices). In other aspects, the one or more orifices do circumscribe the needle element, for example less than 10 degree portion of the needle shaft is not overlapped by an orifice for the length of the needle element shaft.

A wide variety of therapeutic agents may be suitably administered with the present methods and devices, including for example small molecules, peptides, proteins, nucleic acids, nanoparticles, microparticles, gels, biomaterial-drug conjugates, and/or controlled release systems.

Methods for the delivery of an agent are also disclosed. In particular, the methods involve delivery of agents to the eye to treat a variety of ocular conditions such as, for example, retinal detachment, vascular occlusions, proliferative retinopathy, diabetic retinopathy, inflammations such as uveitis, choroiditis and retinitis, degenerative disease, vascular diseases and various tumors including neoplasms.

The present needles and devices may be suitably used for accessing tissues other than ocular tissue, such as epidural spaces, peritoneal spaces, or subcutaneous, including to provide for dermal injections, such as to administer dermal filler materials to a face or other area of a subject. In such methods, a tangential injection is suitably utilized, i.e. where the injection is made at angle (e.g. 1, 3, 5, 10, 15, 20, 30, 40, or 50 degrees) offset with respect to a center of the patient site as disclosed herein for a tangential injection to ocular tissue.

As referred to herein, unless indicated otherwise, the term “fluid” or “fluid composition” includes for example a variety of flowable materials and admixtures including a solution, suspension (e.g. fluid phase with solid phase), or a gel and the like.

Other aspects of the disclosure are disclosed infra.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates gene delivery using subretinal and intravitreal injection according to the related art;

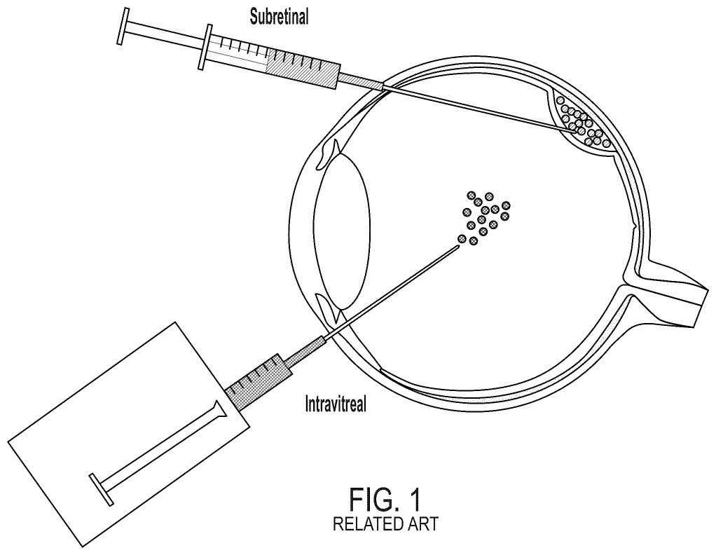

FIG. 2 illustrates theoretical distribution from the subretinal delivery in FIG. 1;

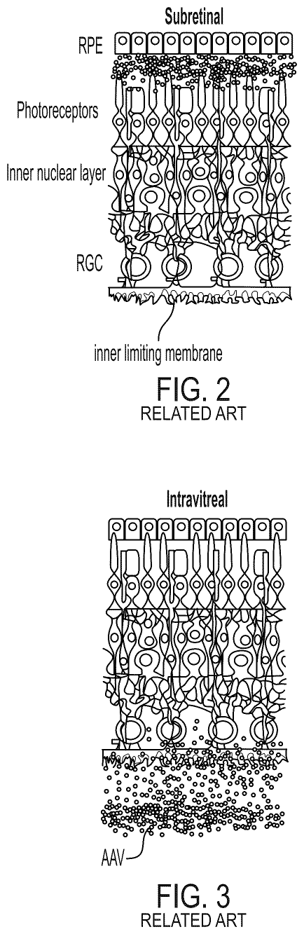

FIG. 3 illustrates theoretical distribution from the intravitreal delivery in FIG. 1;

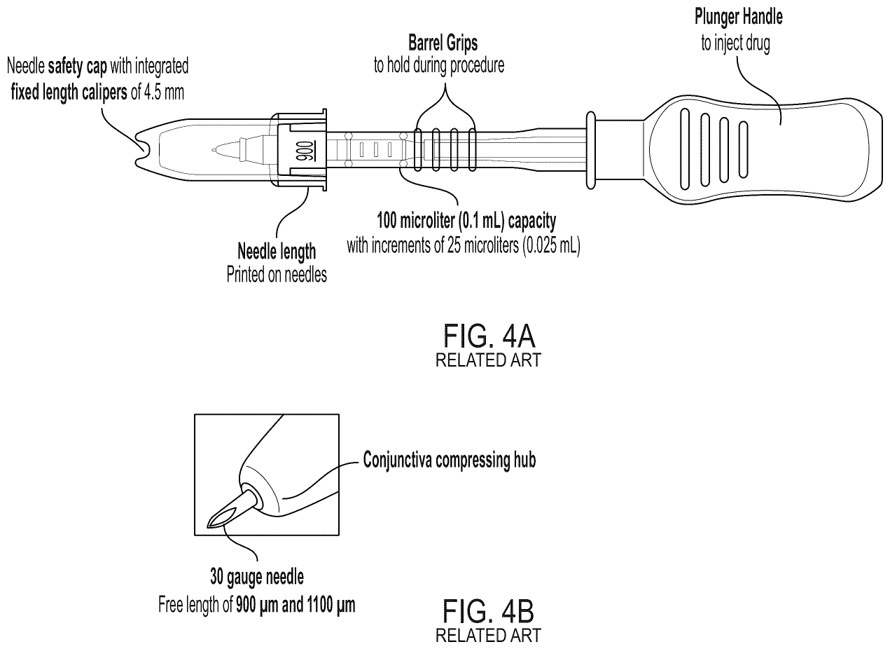

FIGS. 4A-4B illustrate a microinjector according to the related art;

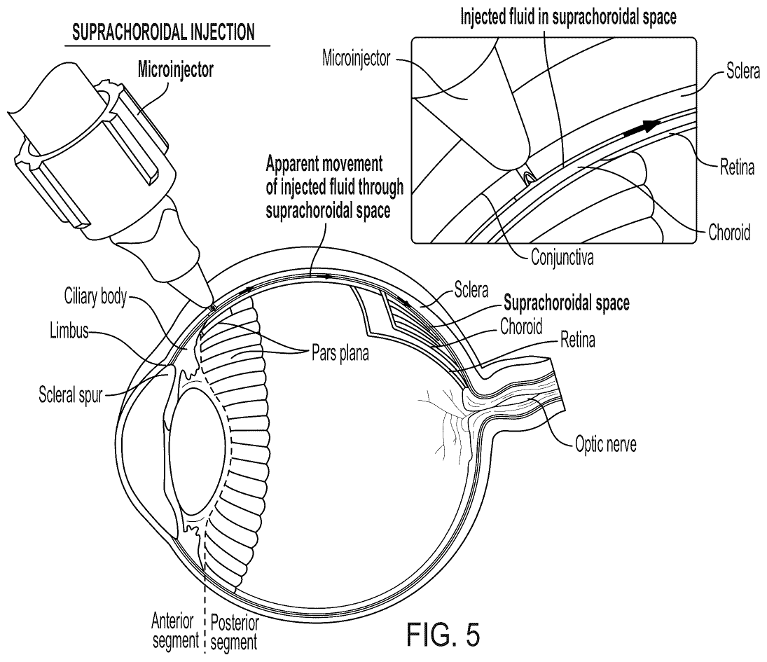

FIG. 5 illustrates the use of the microinjector in FIG. 4A for suprachoroidal delivery of therapeutics;

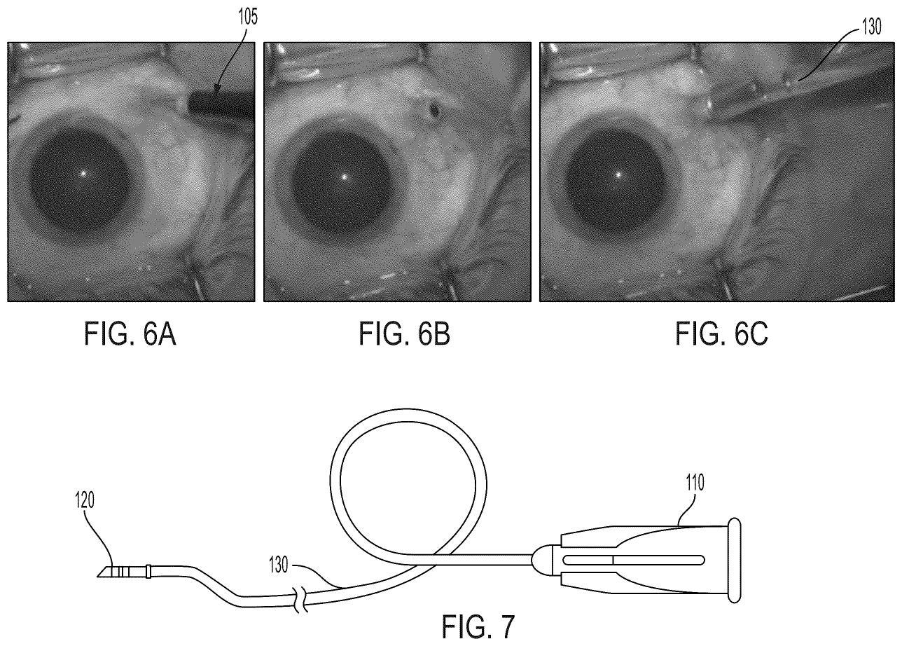

FIGS. 6A-6C illustrate the injection of therapeutics using a trocar cannula according to an exemplary embodiment of the present disclosure;

FIG. 7 illustrates an ocular infusion cannula according to an exemplary embodiment of the present disclosure;

FIG. 8 illustrates the tangential angle of administering the therapeutic agent according to an exemplary embodiment of the present disclosure;

FIGS. 9A-9C illustrate insertion tips according to an exemplary embodiment of the present disclosure;

FIGS. 10A-10B illustrate the surgical instrument according to an exemplary embodiment of the present disclosure;

FIGS. 11A-11B illustrate the surgical instrument according to another exemplary embodiment of the present disclosure;

FIGS. 12A-12D illustrate the surgical instrument according to another exemplary embodiment of the present disclosure; and

FIG. 13A-13D show preferred device designs with pencil point tips.

FIGS. 14A-14B show photographs of devices of FIG. 13A.

FIGS. 15A-15D show several bevel needle configurations. FIG. 15(A): back bevel;

FIG. 15(B): spear bevel; FIG. 15(C) lancet bevel; and FIG. 15(D) spatula bevel.

FIGS. 16A-16C illustrate a comparison of administration of non-viral gene delivery vectors to the SCS using the method of the present disclosure to that of the related art.

FIG. 17A shows an image of an eye following tangential injection with a preferred device (pencil point trocar design). FIG. 17B shows a further image of a frozen block of the eye following injection.

FIG. 18A shows images of an eye following tangential infusion (200 μL/min) with a preferred device (pencil point trocar design). FIG. 18B shows a further image of a frozen block of the eye following fluid infusion.

FIG. 19A shows images of an eye following tangential infusion (100 μL/min) with a preferred device (pencil point trocar design). FIG. 19B shows a further image of a frozen block of the eye following fluid infusion.

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes will be determined in part by the particular intended application and use environment.

### DETAILED DESCRIPTION

The presently disclosed subject matter will be described more fully hereinafter with reference to the accompanying drawings, in which some, but not all embodiments of the inventions are shown. The presently disclosed subject matter may be embodied in many different forms and should not be construed as limited to the embodiments set forth herein; rather these exemplary embodiments are provided so that this disclosure will satisfy applicable legal requirements. Indeed, many modifications and other exemplary embodiments of the presently disclosed subject matter set forth herein will come to mind to one skilled in the art to which the presently disclosed subject matter pertains, having the benefit of the teachings presented in the foregoing descriptions and the associated drawings. Therefore, it is to be understood that the presently disclosed subject matter is not limited to the specific embodiments disclosed and that modifications and other exemplary embodiments are intended to be included within the scope of the appended claims.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The methods discussed herein below provide a minimally invasive approach to controlled administration of gene carriers and other therapeutic agents to the retina and retinal pigmented epithelium (RPE), for example, to the suprachoroidal space (SCS). The types of therapeutic agents may include nanoparticles, microparticles, gels, biomaterial-drug conjugates, drug solutions, suspensions, and other particulate systems. These types of therapeutic agents enable sustained delivery of small molecules or biologics.

Additionally, the surgical instrument described herein is specifically operated at angle for a tangential injection into the eye. The tangential angle is offset (not coincident) from a perpendicular center line of the eye (the eye center line being a line extending outward from the eye that would be perpendicular to the plane parallel to the eye length that is formed by the outer reaching eye surface). This tangential injection angle suitably may be for example 0.5 to 45 degrees, or 1 to 45 degrees or 3 to 30 degrees or 3 to 10, 15, 20, 25 or 30 degrees) from a perpendicular center line of the eye.

Once the eye is penetrated, the therapeutic agent may be administered through the penetration site. The administering of the therapeutic agents provides a slow, controlled infusion into the SCS of the eye, allowing the agent to be injected at a particular location within the eye where needed yet can also be spread to cover the full retina. The controlled infusion also allows for administration of increased volumes of therapeutic over time as injected fluid is simultaneously absorbed and circulated over the same time period. By injecting the therapeutic agent at the tangential angle, the risk of inadvertent intravitreal injection and intraocular pressure is decreased compared to a perpendicular injection.

In particular, the tangential administration of a composition comprising the therapeutic agent in a given time period results in pressure within the SCS that is substantially reduced compared to the pressure within the SCS that results by a perpendicular administration of the same volume of the composition within the same time period. The tangential administration results in SCS pressure that is about 5-20 percent less relative to the perpendicular administration. Accordingly, the present disclosure provides a simplified, non-invasive method for ocular procedures such as ocular gene therapy which thus optimizes therapeutic efficacy.

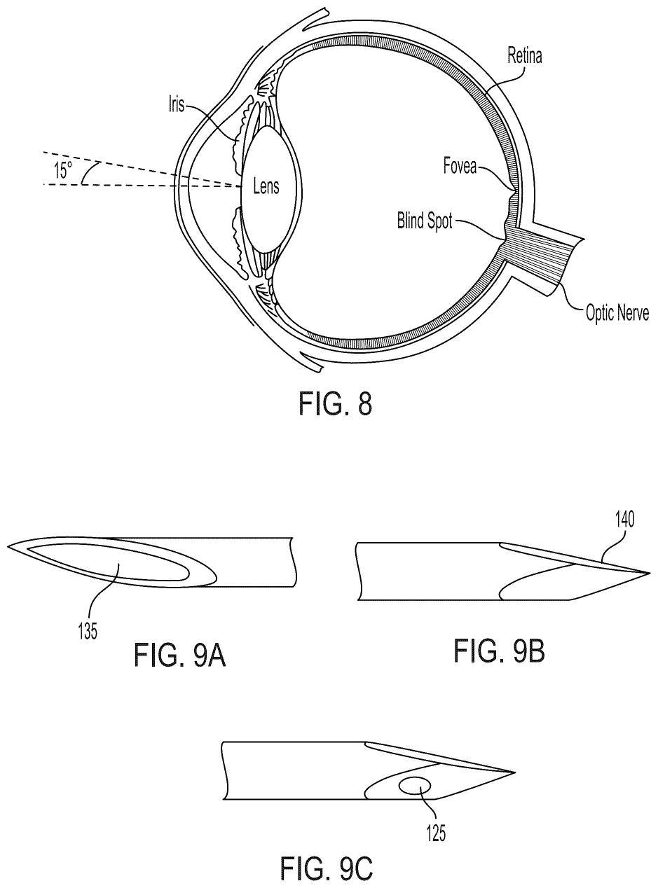

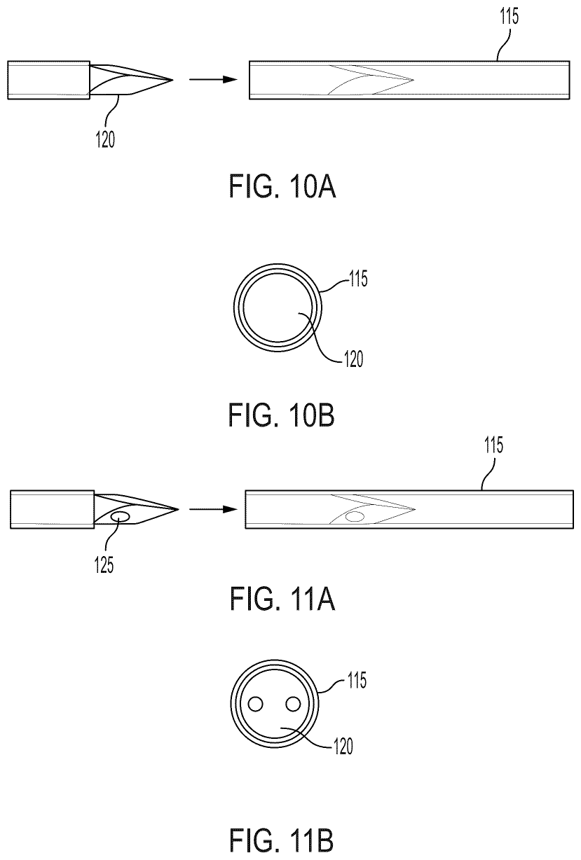

FIGS. 6A-6C and 7, illustrate the surgical instrument that is operated for performing the method of the present disclosure. In particular, the surgical instrument may include a cannula 105 that is connected to a handle 110 of the instrument and through which therapeutic agents are delivered into the eye via a continuous and controlled infusion. A sheath 115 in which the cannula is housed has a diameter that is greater than a diameter of the cannula. The sheath 115 encloses an insertion tip 120 that is formed at a distal end of the cannula 105 to penetrate the tissue of the eye at a tangential angle. The cannula may be a trocar cannula as shown in FIGS. 6A-6C or an infusion cannula as shown in FIG. 7, however, the present disclosure is not limited thereto. Notably, although FIGS. 6A-6C illustrate a perpendicular injection, the present disclosure is specifically related to a tangential injection.

Herein below a description will be provided of each of a trocar cannula and an infusion cannula and the method of tangentially administering the therapeutic agents using each of the cannulas.

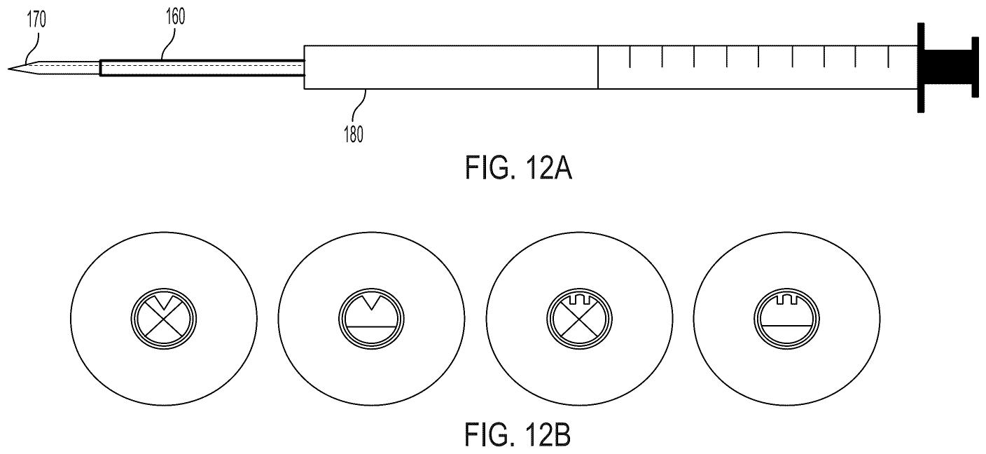

FIG. 6A illustrates the trocar cannula 105 with the insertion tip at the distal end thereof being a closed end of the cannula. Since the insertion tip is a closed end of the cannula in this embodiment, the incision into the eye may be first performed by sclerotomy as a preparation procedure and then the insertion tip is inserted into the eye at a tangential angle. That is, the user may grasp the handle of the instrument and orient the insertion tip at a tangential angle relative to the eye and move the insertion tip into the incision. This tangential angle may be about 5 to 30 degrees, but preferably, 0-15 degrees, as shown in FIG. 8 which illustrates a perpendicular center line. FIG. 8 illustrates the perpendicular center line of the eye and shows a 15 degree angle therefrom as an example. Notably, the present disclosure is not limited to the particularly listed angles. Various techniques for controlling this angle will be described herein below which allows the user to be notified if the angle is changed and ensures higher accuracy.

The sheath is then deployed using a manipulator and then the cannula 105 is released into the eye, as shown in FIG. 6B. That is, the cannula remains indwelling once separated from the other components. The present disclosure is not limited to using a manipulator to deploy the sheath and any other mechanism may be used to enable such deployment. Thereafter, as shown in FIG. 6C, infusion tubing 130 is connected to the cannula 105 that is left in the eye to deliver the therapeutic agents or gene carriers therethrough and into the eye. The infusion tubing is connected to a controlled flow system to thus provide controlled flow rates of the therapeutic agents.

According to another exemplary embodiment and as shown in FIG. 7, the cannula may be an infusion cannula. In this embodiment, the insertion tip 120 may be a needle-like member that directly penetrates the eye. Accordingly, a preparation procedure would not be required as the cannula tip itself is able to penetrate the eye. The various configurations of the insertion tip and cannula will be described herein below. Additionally, in this configuration, the cannula is not left within the eye. Instead, the infusion tubing is connected directly to the cannula.

Particularly, the insertion tip penetrates the eye at a tangential angle of about 0-15 degrees and then the sheath is deployed. The therapeutic agents are delivered directly via the infusion tubing already connected to the cannula. The cannula may also be bent at a particular angle to facilitate the positioning of the cannula on the eye. That is, as shown in FIG. 7, the end of the cannula may be bent to help a user position the cannula against the eye at the tangential angle. In both embodiments of FIGS. 6A-6C and FIG. 7, the insertion tip may specifically penetrate the suprachoroidal space of the eye.

Additionally, in both of the above-described embodiments, the infusion tubing may be connected to a controlled flow system. For example, the infusion tubing may be connected to a pump, vitrectomy equipment, or the like. This allows a flow rate of about 0.001-250,000 μL/h to be administered. A flow rate of less than about 90,000 μL/h is preferable, and more preferably, about 50-8,000 μL/h, or 50-7,000 μL/h, or 50-6,000 μL/h. As another technique for controlling the flow rate, the cannula, syringe, or fluid depot may include numerous gradation which serve to further slow the fluid infusion. The controlled infusion of the therapeutic agents allows for varied coverage of the SCS area of the eye. For example, 80-100% of the SCS area, 50-80% of the SCS area, or a more local portion of the <50% of the SCS area. In another embodiment, the infusion of the therapeutic agents may be combined with an anterior chamber tap that facilitates delivery of larger volumes of the agents at increased flow rate of injection.

According to another exemplary, the infusion of therapeutic agents may be proceeded by or concurrent with a balloon or other types of mechanical expansion components for the SCS. The infusion may also be proceeded by or concurrent with collagenase or other chemically-medicated mechanisms of SCS expansion. Additionally, the therapeutic agents may be infused in multiple quadrants of the eye either in a sequential manner or simultaneously to maximize the payload volume and distribution.

Furthermore, the present disclosure provides various embodiments to secure the cannula to the tissue site of the eye. In one example, the outer surface of the tip of the cannula may be formed with nano- or micro-topographic features. These obstacles may surround the external surface of the cannula or sheath, or be formed as ridge-like barriers to prevent the cannula tip from slipping out of position. In addition, the cannula or handle many have attachments that facilitate connection to the patient's speculum. As another example, the cannula may include flanges or wings that expand to hold the cannula in place. The wings may also be adhesive wings that support the attachment to the eye or speculum. Alternatively, the cannula may include an overcap attachment activated upon insertion into the eye. A seal may also be provided at the cannula to eliminate dead space and prevent air infusion into the eye. The present disclosure is not limited to these features and any other connection that enables attachment to the eye may be used. Notably, the use of additional securing features ensures that the tangential angle to the eye is maintained during the administration of the therapeutic agents.

As another feature that ensures angle alignment, the cannula may include various sensors such as piezoelectric sensors. For example, the sensor may output a signal if the angle of the cannula changes relative to the eye. The sensor may also output a signal that indicates when the tissue of the eye has been penetrated and when the tip of the cannula is within the SCS. As another example, the sensor may output various signals related to the amount of payload being administered. These sensor output signal ensure insertion accuracy as well as enable reproducibility. The signals may be transmitted to an external device or to the surgical instrument itself and output via an interface for a user. In this manner, the pressure signals that indicate presence within the SCS may trigger fluid administration via pump.

In other embodiments, the syringe configuration itself could be modified to only allow for fluid outflow when resistance at the needle tip is reduced upon entry into the SCS. For example, through use of the needle embodiments disclosed here in a fluid and moveable configuration in combination with a pushing-plunger, needle-plunger, and mechanical stop. A physical stop or barrier prevents penetration of the needle too far into the tissue. As the syringe plunger is advanced, the increased pressure on the fluid advances the needle until it reaches the target cavity or potential space at which time fluid is released and the pressure on the fluid inside of the syringe is decreased, preventing further advancement of the needle.

Notably, the present disclosure is not limited to these particular output signals of the sensors and various other output signals may be provided to improve the accuracy of the instrument use.

In a further aspect, a device may comprise one or more visual markers to aid manipulation and location of the device. For instance, a device may include one or preferably multiple several visual markers in order to ensure tangential administration at the appropriate insertion depth. In one example, the tip of the needle or cannula is colored or stained in order to improve visualization during and after insertion. As another example, a colored marker, groove, or ridge may be incorporated into the cannula, for example, between 1 and 4 or 1 and 5 mm from the tip in order to allow for appropriate length of insertion. As another example, a physical stop could also be connected to the needle or cannula, such as an external shaft or cannula. In addition, a level or other method of visual angle measurement, may be incorporated into the handle of the surgical instrument, allowing the clinician to monitor the angle of the instrument at all times. Other visual signals and other markers also may be provided to optimize insertion angle and depth.

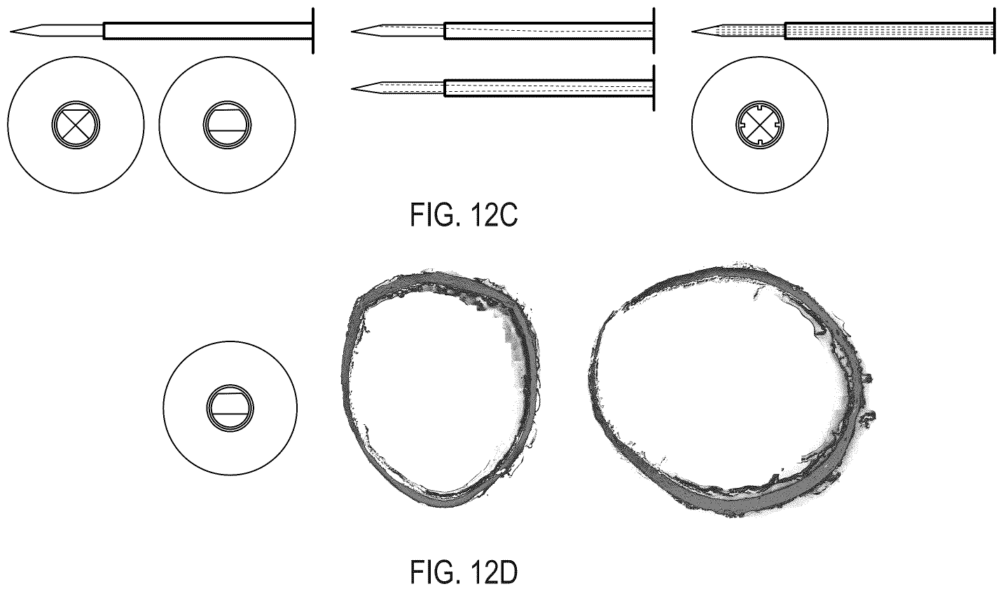

FIGS. 9A-9C illustrate variations of the insertion tip of the cannula. In particular, the insertion tip may be varied in gauge, length, fluid outlets, and shape. For example, the length of the insertion tip may range from about 1-5 mm and the gauge may be about 25-30 G for the trocar cannula and about 25-33 G for the infusion cannula. FIG. 9A illustrates an insertion tip with a single opening 135 providing a hollow flow path therethrough. FIG. 9B illustrates a tapered insertion tip and FIG. 9C shows a tapered insertion tip 140 with one hole 125 on at least one tapered surface for the flow of therapeutic agent. The cannula tip (insertion tip) may also be formed as a closed end for the trocar cannula. In this case, in certain embodiments, the tip may be manually or automatically retracted via a sliding, spring, or other mechanism, allowing for therapeutic administration in the space between the tip and the sheath. As another example, the cannula tip may be formed as a lancet, menghini, diamond, pencil point, or other design. In additional embodiments, the cannula tip of any such design is shaped asymmetrically and inserted into the SCS such that the tip veers towards the sclera during advancement in order to prevent inadvertent intravitreal injection. Notably, the present disclosure is not limited to these particular designs and other shape and fluid outlets are contemplated. The flow outlets shown in the drawings are optional.

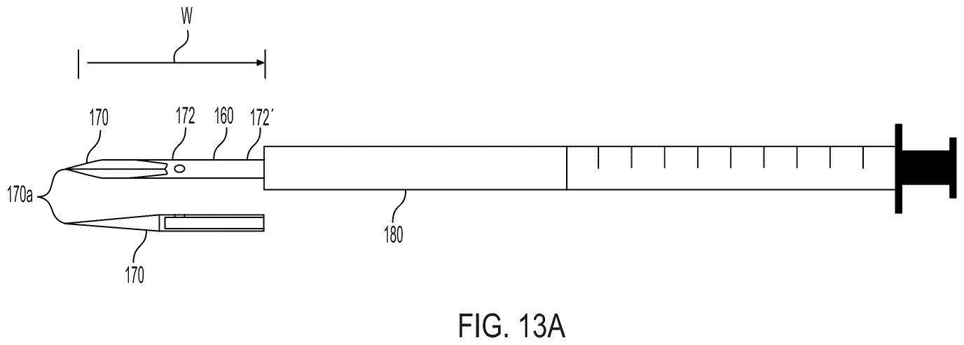

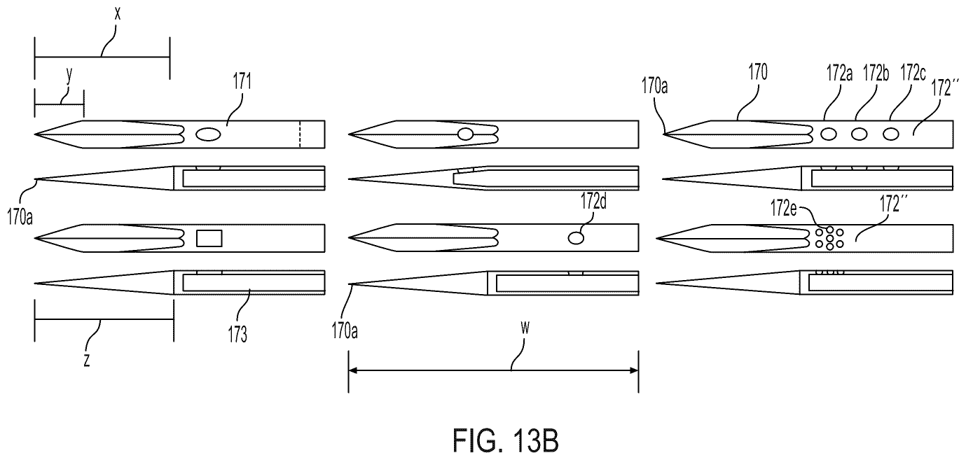

FIGS. 10A-10B and 11A-11B further illustrate the cannula design according to an exemplary embodiment of the present disclosure. FIGS. 10A-10B correspond to the insertion tip structure of FIG. 9B and FIGS. 11A-11B correspond to the insertion tip structure of FIG. 9C. The sheath 115 shown in the figures is constrained by the outer diameter of the insertion tip 120 which may be about 0.35-0.37 mm. As described above, a manipulator may be maneuvered or otherwise engaged to deploy the sheath during the operation of the surgical instrument. The manipulator may be provided in different forms on the handle of the instrument. For example, the manipulator may be in the form of a button, slider, wheel, or the like. The deployment of the sheath then enables the extension into the SCS of the eye.

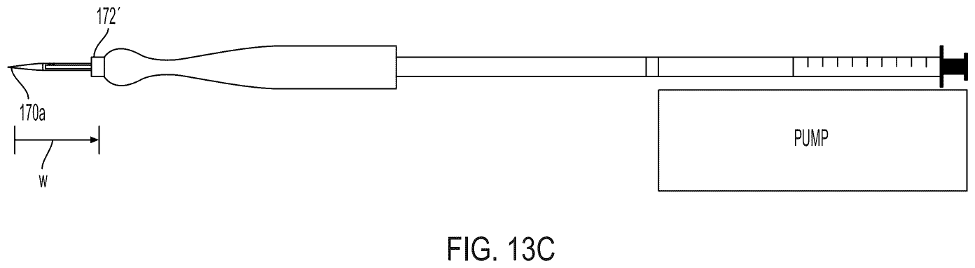

FIGS. 12A-12C further illustrate variations of the cannula and tip design according to exemplary embodiments of the present disclosure. FIG. 12A corresponds to a cannula and handle designed for one-step insertion into the SCS and delivery of therapeutics. In this case, the cannula 180 is directly connected to a syringe 160 with tip portion 170; however, in other embodiments, it may be connected to tubing, a handle, an adaptor/connector, or other means of fluid communication and delivery. FIG. 12B corresponds to potential cross-sections of the tip design in FIG. 12A that allows for facile insertion into the SCS while providing a channel for fluid flow. FIG. 12C corresponds to variations in the tip design that provide fluid channels of varying geometries. Through modulation of the tip design, a channel(s) is created between the insertion tip and the cannula, which is connected directly to the therapeutic depot, and which may be utilized to further control the flow rate and administration of therapeutics into the SCS. 1-3 mm of the cannula will penetrate the SCS and be retained within the SCS in order to prevent leakage during administration. Notably, these embodiments can be inserted such that the fluid channel delivers therapeutics against the scleral border of the SCS rather than the choroidal border, which may allow for safer and more comfortable delivery of therapeutics. In other embodiments, the channels may be formed circumferentially around the insertion tip. Notably, the present disclosure is not limited to these particular designs and other insertion tips and fluid channels are contemplated. FIG. 12D corresponds to application of a specific embodiment from FIG. 12C for tangential injection of fluorescein isothiocyanate-labeled dextran into the suprachoroidal space of two ex vivo porcine eyes. The 25 G-sized embodiment was able to successfully penetrate the sclera, enter the suprachoroidal space, and deliver 100 μL of fluid in a controlled manner against the sclera while retained in the suprachoroidal space. The distribution of labeled dextran was determined by taking images from sections around the entire globe and stitching them together. Notably, in both porcine eyes, the labeled dextran is confined to the suprachoroidal space and extends around the globe. This implies that the one-step injection was performed safely (without penetration into the intravitreal space) and successfully (broad distribution circumferentially throughout the suprachoroidal space).

In regards to the therapeutic agents that are administered, various properties are considered in the present disclosure. For example, both viral and non-viral nucleic acid delivery vectors; prophylactic and therapeutic moieties, including small molecules, peptides, proteins, drug conjugates, cells, and their combinations in a variety of forms such as in solution, suspension, nano- or microparticles, gels, liposomes, polymers, dendrimers, biomaterials, implants, and the like; and preferably in an isotonic or hypotonic solution if delivered as a liquid.

In various preferred devices, a needle of a trocar system may be closed-ended stiletto, and in certain configurations a tapered (preferably pointed tip) and suitably including a beveled face and taper tip, which are placed inside the orifice of the main needle tube. The outer cannula builds the trocar suitably may have a 20-30 gauge diameter, such as a 22, 23, 24, 25, 26, 27, 28 or 29 gauge diameter.

A beveled face configuration may include an angled surface on the needle shaft (preferably a sharpened stiletto design) to form a slanting edge at the needlepoint, facilitating an atraumatic penetration through the sclera into the human globe. A preferred needlepoint known as a lancet or diamond point is one of the sharpest points available for any medical needles.

In a particularly preferred design a needlepoint may include multiple bevel cuts, for example three bevel cuts: The primary bevel, which is the surfaced as a result of grinding the tube at a specific angle α, and the two-side bevels, which are secondary grind angle β on each side of the primary bevel to form the cutting edge and a sharp needlepoint. The bevel length is by definition the longest distance of a bevel, measured from the tip of the needle to the most proximate area of grinding behind the heel. The side bevel length is measured between the juncture of side bevel, with the outside surface of the angled surface, and the tip of the needle.

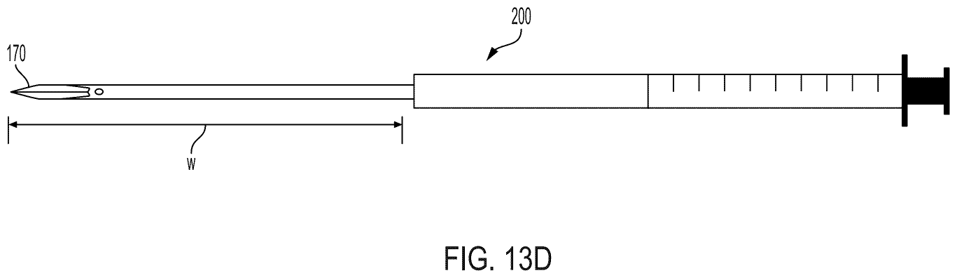

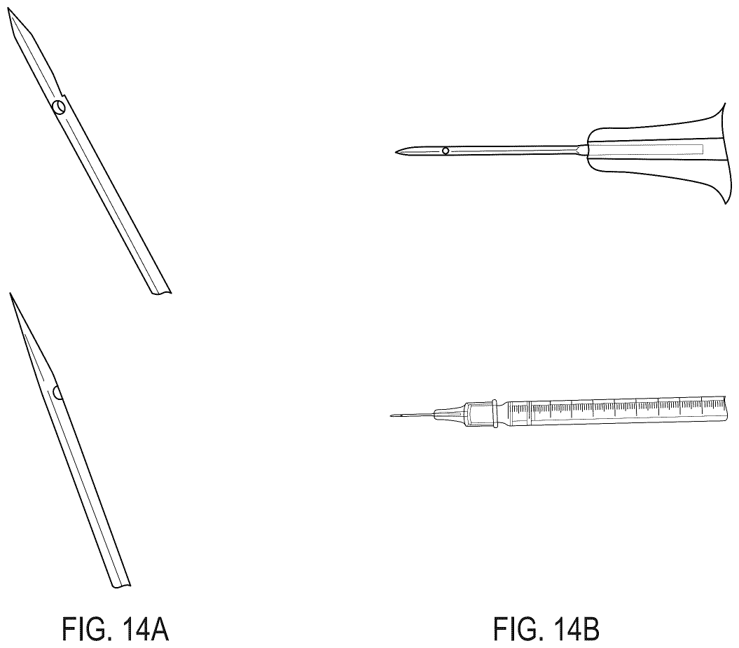

Particularly preferred devices are depicted in FIGS. 13A-13C and 14A-14B. FIG. 13A schematically depicts a so-called pencil point trocar embodiment, which can allow for a one-step insertion and fluid delivery into a subject eye, particularly the suprachoroidal space. The positioning of the outlet allows for flow to be directed against the scleral border of the SCS rather than the choroidal border, which may allow for safer and more comfortable delivery of therapeutics. In 13A, cannula 180 is connected to syringe 160 with tip portion 170. The connection may be suitably made for example via a conventional luer lock mechanism or other mechanism suitable for connection of the cannula to a medical syringe. One or more markers (for example, may be radiopaque or visual marker) may be placed along the length of the needle to indicate length of needle inserted into the suprachoroidal space.

FIG. 13B depicts exemplary needle embodiments, including variation of outlet dimensions and geometry, positioning of the outlet along the length of the needle (such as to position fluid flow against the scleral border of the SCS), and number of outlets. Needle diameter (OD) may suitably vary somewhat widely and in preferred aspects may range from 25-30 gauge and may be for example 25, 26, 27, 28, 29 or 30 gauge. Again, one or more markers such as the depicted 172a, 172b, 172c, 172d and 172e may be positioned along a needle (such as 171 and 173) to facilitate positioning of the device within a patient's eye. In exemplary embodiments, the distal edge of the outlet may be positioned 2 mm from the tip of the trocar blade and a stop, sheath, or marker may be positioned 1.5 mm from the proximal edge of the outlet, allowing for insertion of 4 mm of the needle into the SCS. This design may further limit the risk of fluid reflux.

FIG. 13B shows varying configurations of the preferred pencil point end portions, such as in the device shown in upper left of FIG. 13B where the end portion tapers to as point (for a distance y) that is a portion of the end length x, for instance where distance y is up to or less than 10, 20, 30, 40, 50, 60 or 70, 80, 90 or 95 percent of the length x. In the device shown in the lower left of FIG. 13B, the tip portion tapers to a point for the entire distance z.

In certain preferred devices, the distance x of a needle element as exemplified in FIG. 13B suitably may be from 0.3 or 0.4 mm to 5 mm, more typically 0.5, 0.6, 0.7 or 0.8 mm to 1, 2, 3 or 4 or 5 mm. In certain preferred devices, the distance y of a needle element as exemplified in FIG. 13B suitably may be from 0.3 or 0.4 mm to 5 mm, more typically 0.5, 0.6, 0.7 or 0.8 mm to 1, 2, 3 or 4 or 5 mm.

In certain preferred devices, the distance z of a needle element as exemplified in FIG. 13B suitably may be from 0.3 or 0.4 mm to 5 mm, more typically 0.5, 0.6, 0.7 or 0.8 mm to 1, 2, 3 or 4 or 5 mm.

In FIG. 13, needle insertion length W is depicted as the length from the needle tip 170 to the interface with cannula 180. As discussed, the insertion length suitably may be at least or up to e.g. 0.25, 0.5, 0.75, 1.0, 1.25, 1.5, 1.75 or 2.0 mm. The insertion length of a needle element is the length of the needle element that is configured for preferred insertion into patient tissue such as ocular tissue.

In certain preferred devices, a needle insertion length can be defined as the needle length ending from the needle tip (such as 170a shown in FIGS. 13A and 13B) to a point along the needle shaft distal to the needle tip 170a such as distal end of the most distal orifices such as a point 172″ shown in FIG. 13B or where a luer or cannula can be as a stop such as point or interface 172′ exemplified in FIGS. 13A and 13C. The insertion length also can be defined as a specified length ending from needle tip 170a (such as length W shown in FIGS. 13A, 13B, 13C and 13D).

FIG. 13C shows a pencil point trocar embodiment integrated into a fluid infusion system that includes a handle, tubing, and infusion pump to allow for controlled fluid infusion into the suprachoroidal space. Different types of pumps may be utilized, including syringe infusion pumps, disposable pumps, elastomeric pumps, or small volume pumps. Different types of pencil point trocar embodiments could be configured into this system, including those described in FIGS. 13A and 13B. FIG. 13D shows another preferred needle device 200 with needle and tip portion 170.

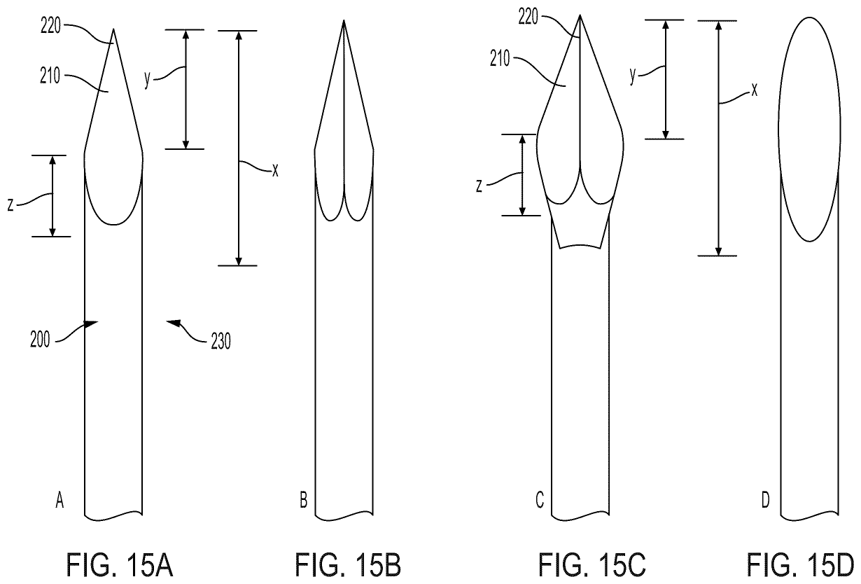

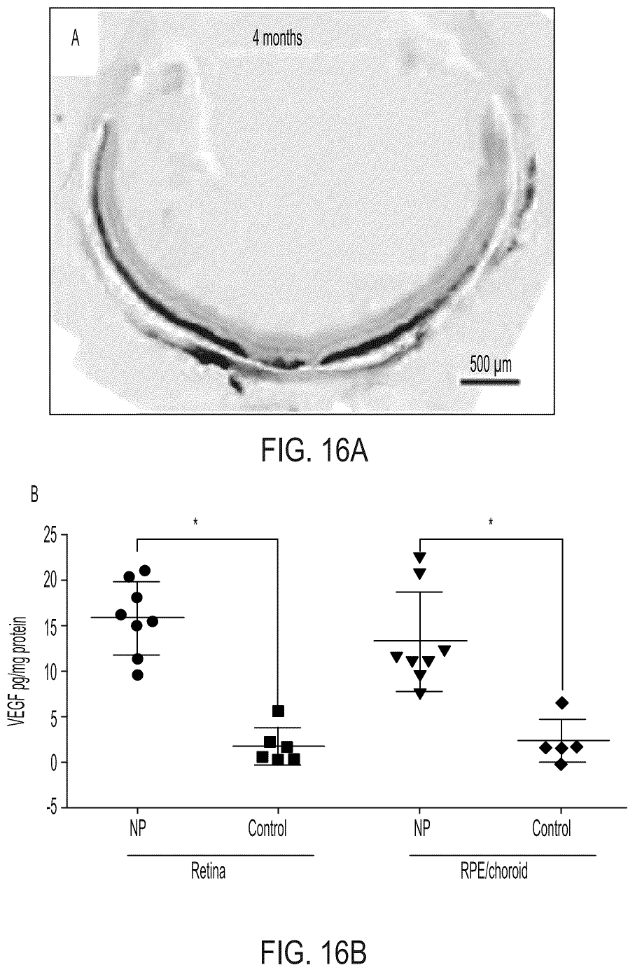

FIGS. 14A and 14B are photographs of the devices depicted in FIGS. 13A-13D. In FIGS. 15A-15D, various preferred beveled needle designs are shown, in particular a “back” bevel configuration in FIG. 15A, a “spear” bevel configuration in FIG. 15B, a lancet bevel configuration in FIG. 15C and a “spatula” bevel configuration in FIG. 15D.

In the configuration shown in 15A, an angled surface on the shaft of the stiletto forms a slanting edge at the sharpened needlepoint. The needle tip of the back bevel is sharply angled and has an edge behind the bevel. In certain preferred designs, the stiletto may have a secondary grind on the opposing sides of the primary grinds. In certain preferred designs, the back bevel needle is smaller compared with that of the lancet needles.

In the configuration shown in FIG. 15B, the depicted preferred design depicts the needle type more acutely angled in a triangular shape. The spear bevel design suitably may include an angled surface on the shaft of the stiletto to form a pyramid-like slanting triangular needlepoint. In certain preferred designs, the needle tip of the spear bevel may be more acute that may be provided by a secondary grind on the sides of the primary grinds.

In the configuration shown in FIG. 15C, the lancet bevel has a tip or end portion that that expands from needle shaft 230 and an edge behind the primary bevel. The lancet bevel point or diamond point has a needle tip with three bevels which can be provided a three-grind needlepoint formed from a primary grind and two secondary grinds. In the depicted design, the broadest part of bevel diameter is greater (e.g. at or up to 1, 2, 5, 8, 10, 20, 30, 40 or 50 percent greater) compared with the tip of back. However, it becomes tapered by 45° because of a second grind giving it the appearance of a diamond-shaped tip. The bevel face of the lancet needle is bigger than that of the back needle.

In the configuration shown in FIG. 15D, the depicted spatula bevel has a round frontier which suitably can be provided by a backcut method. The spatula bevel with no sharp needle tip is suitably provided in combination with a secondary bevel angle. The bevel is the beveled surface on a shaft to form a smooth closed-ended slanting junction with an oval tip to facilitate an atraumatic penetration, which appears sometimes more cumbersome to create a sclerotomy.

Generally preferred needle elements may be fabricated from sterilizable, biocompatible materials, including metals and polymeric materials, preferably stainless steel as well as various polymeric materials. Needles may also be coated to further reduce resistance.

As discussed, preferred needle elements include those that have a size of 25-30 gauge including 25 G and 27 G, which may provide an inner diameter of 260-159 μm and a range of outer diameter of 514.4-311.2 μm.

Needles of other cross-sectional dimensions also will be suitable and preferred. For instance, needles suitably may have small inner diameters such as less than or up or less 150, 140, 130, 120, 100, 100, 90 or 80 μm. In certain systems, those smaller inner diameters suitably may be utilized in needle configurations with outer diameters as discussed above, i.e. in an outer diameter range of about 520 or 514 to 300 or 311 μm.

Needles of varying lengths may be suitably employed. In certain embodiments, for insertion into the SCS, a needle length (from entry point to distal end) suitably may be e.g. 0.5 to 5, 6, 7, 8, 9 or 10 mm, may typically 1 to 5 mm, or 2 to 4 or 5 mm. Needles of length of up to or at least 0.5, 1, 1.5, 2, 2.5, 3, 3.5, 4.4.5, 5, 5.5, 6, 7 or 8 mm may be suitable, with length of up to or at least 0.5, 1, 1.5, 2, 2.5, 3, 3.5, 4, 4.5, 5 or 5.5 mm being preferred for at least certain applications. In certain systems for SCS insertions, the above needle lengths will be the needle portion that extends within the subject's eye, and the needle overall length can suitably those lengths.

For example, in certain systems, an extended needle length (which can include needle length outside the SCS) can provide for luer hub integration as a component of the needle device. In certain preferred systems, an extended needle length suitably may be from about 0.25 cm to 5, 6, 7 or 8 cm, more typically 0.5 cm to 2, 3, 4 or 5 cm or 0.5 cm to 1, 2 or 3 cm.

Preferred needle devices suitably may have one or more preferably multiple outlets. Multiple outlets may be preferred to reduce risk of clogging and be less traumatic to ocular tissue. Suitable outlet dimensions vary. For certain preferred needle devices, such as 25-30 G and 1-5 mm length of ocular insertion, a maximum width and length of an outlet could be up to 514.4 μm (including up to 400, 450, 500, 510) and 3 mm (including up to 0.25, 0.5, 1, 2, 2.5, 2.8 or 3 mm), respectively. Outlets may be formed in a range of shapes fitting these dimensions, including circles, ovals, squares, rectangles, or other continuous and non-continuous shapes with a preference for those that provide sufficient surface area for laminar fluid flow. For certain systems, needle orifices having lengths and/or widths less than about 20, 15, 10, or 5 μm may be less preferred.

Needle orifices may be positioned on a needle device in a variety of configurations, including circumscribing a needle shaft. In certain preferred devices, one or more orifices of the needle may be restricted to one side (i.e. not circumscribing) of the needle thereby facilitating flow direction against one tissue layer. In FIG. 13B, the needle devices with orifices 172a, 172b, 172c and then the device with orifice 172e exemplify configurations where one or more orifices of the needle may be restricted to one side of the needle.

A wide variety of agents may be administered using the present devices, systems and methods, including for example nucleic acids, an antibiotic agent, a beta blocker, a corticosteroid agent and others.

The present devices, systems and methods also may be used to treat a variety of diseases and disorders, for instance, macular degeneration including age-related macular degeneration (AMD), neovascular age-related macular degeneration (NVAMD), retinitis pigmentosa (RP), optic neuritis, infection, uveitis, sarcoid, sickle cell disease, retinal detachment, temporal arteritis, retinal ischemia, choroidal ischemia, choroidal ischemia, ischemic optic neuropathy, arteriosclerotic retinopathy, hypertensive retinopathy, retinal artery blockage, retinal vein blockage, glaucoma, hypotension, diabetic retinopathy, diabetic macular edema (DME), macular edema occurring after retinal vein occlusion (RVO), macular edema, and choroidal neovascularization.

In certain embodiments the systems and methods also may be used to treat a variety of genetic diseases and disorders including Usher Syndrome, Stargardt disease, Leber Congenital Amaurosis, Choroideremia, and Cone-rod Dystrophy.

The surgical instrument and method of operating the same described herein has several advantages over conventional techniques. In particular, the present disclosure provides a minimally invasion intervention that does not require a surgical procedure, thus reducing overall costs and complications associated with such procedures. Additionally, the insertion at a tangential angle is reproducible and enables localized delivery and broader retinal coverage. That is, the surgical instrument described herein provides control of insertion depth and angle which improves reproducibility and reduces the risk of inadvertent intravitreal or subretinal injections. The therapeutic agents or genes are capable of being delivered through controlled, continuous infusion which enables optimal delivery of the maximum volume of agents while reducing the risk of pain or an increase in intraocular pressure.

### EXAMPLES

The following non-limiting examples are illustrative.

### Example 1

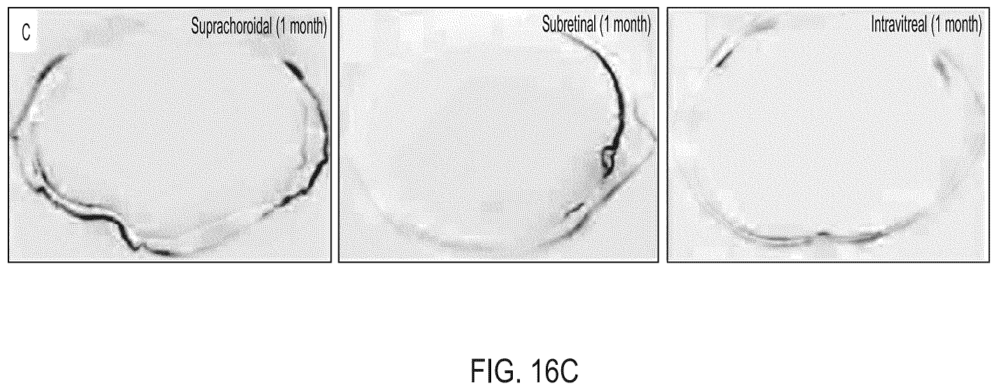

This Example is a comparison of administration of non-viral gene delivery vectors to the SCS in rats using the method of the present disclosure to an evaluation of distribution or viral vectors in non-human primates via SCS microinjector, subretinal, and intravitreal administration methods of the related art. FIG. 16A illustrates results with a method of the present disclosure, the suprachoroidal injection of non-viral gene delivery nanoparticles spread throughout the full retina of rats fourth months after a single injection. FIG. 16B shows that the SCS injection according to the present disclosure results in significant levels of expression of a recombinant protein in the retina and RPE. FIG. 16C shows the distribution evaluation of the methods of the related art. In particular, FIG. 16C shows different patterns of green fluorescent protein (GFP) transgene expression following administration of viral vectors using the method of the related art. As shown, the method provided by the present disclosure results in higher levels of expression of the protein than that of the related art.

### Example 2

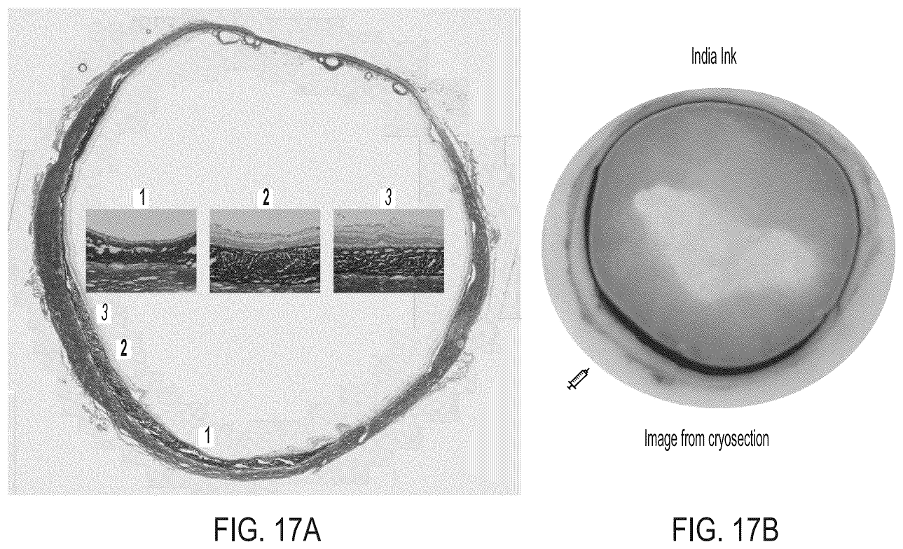

A device having a 25 G pencil point trocar of the configuration shown in FIG. 14A is used. FIG. 17A is a cryosection image of the porcine eye following injection of 150 μL of India Ink from the 25 G pencil point trocar with high magnification images in the center. As shown in FIG. 17A, the ink is fills the suprachoroidal space between the sclera and the RPE and retina. FIG. 17B is a gross image of the frozen section block of the porcine eye following injection. The syringe indicates the site of the tangential injection. The suprachoroidal space is thicker and full of ink at the site of the injection. The ink extends from 4:00 around clockwise to 10:00.

### Example 3

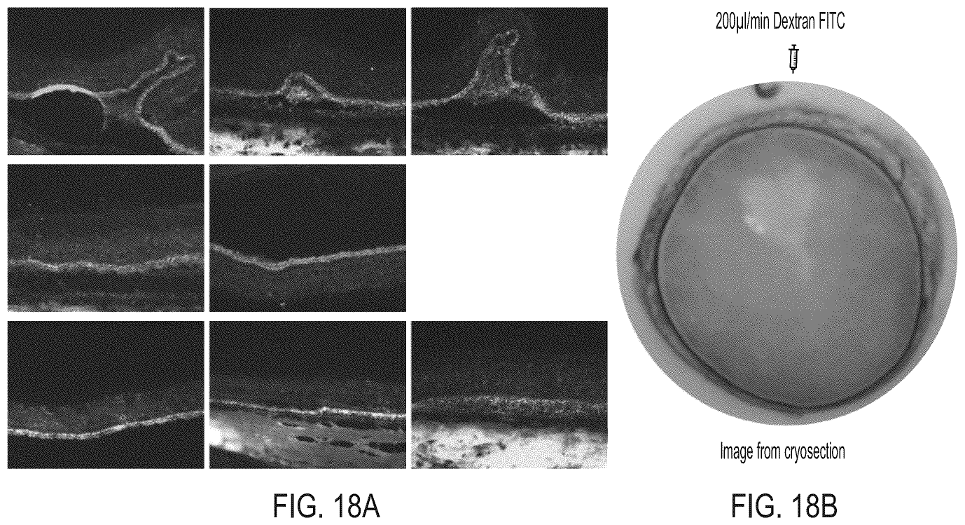

FIG. 18A are high magnification images of sections of a porcine eye following tangential infusion of fluorescein isothiocyanate-labeled dextran at 200 μL/min into the suprachoroidal space via the 25 G pencil point trocar (FIGS. 13A and 14A) showing a thin layer of fluorescein isothiocyanate-labeled dextran under the retina and RPE. FIG. 18B is a gross image of the frozen section block of the porcine eye following fluid infusion. The syringe indicates the site of the tangential injection. The suprachoroidal space is not thickened as much near the injection site in comparison to direct injection (FIG. 16, Example 1 above), and the fluorescein isothiocyanate-labeled dextran is distributed all of the way around the circumference of the eye.

### Example 4

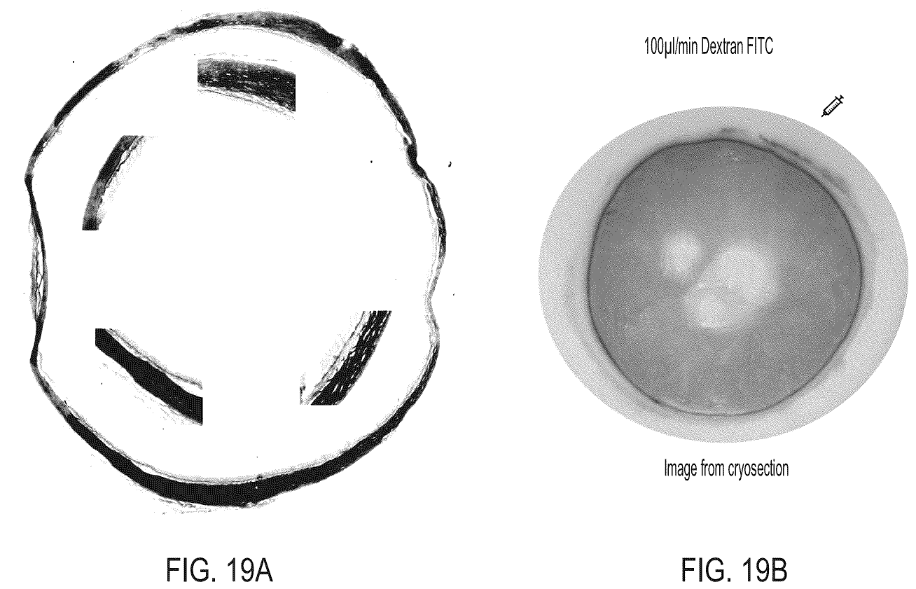

FIG. 19A shows fluorescence image of section of a porcine eye following tangential infusion of fluorescein isothiocyanate-labeled dextran at 100 μL/min into the suprachoroidal space via 25 G pencil point trocar (FIGS. 13A and 14A) with high magnification images in the center showing a thin layer of fluorescein isothiocyanate-labeled dextran under the retina and RPE. FIG. 19B is a gross image of the frozen section block of the porcine eye following fluid infusion.

In FIG. 19B, the syringe indicates the site of the tangential injection. The suprachoroidal space is not thickened as much near the injection site in comparison to direct injection (FIG. 15), and the fluorescein isothiocyanate-labeled dextran is distributed all of the way around the circumference of the eye. Thus, controlled infusion of therapeutics via exemplary embodiments may allow for improved safety and distribution.

### Example 5: Treatment of Patient with Macular Degeneration

A patient is diagnosed as suffering from age-related macular degeneration.

A device as shown in FIGS. 13A and 14A is loaded with a fluid composition containing bevacizumab at a dose of 3 mg. A tangential injection is made to the suprachoroidal space (SCS) of the patient's eye and the drug composition administered.

The many features and advantages of the disclosure are apparent from the detailed specification, and thus, it is intended by the appended claims to cover all such features and advantages of the invention which fall within the true spirit and scope of the disclosure. Further, since numerous modifications and variations will readily occur to those skilled in the art, it is not desired to limit the invention to the exact construction and operation illustrated and described, and accordingly, all suitable modifications and equivalents may be resorted to, falling within the scope of the disclosure.

1. A method of treating an eye of a subject, comprising:
tangentially administering a therapeutic agent to the eye to infuse the therapeutic agent to a suprachoroidal space (SCS) of the eye.

2. The method of claim 1, wherein the therapeutic agent is administered to the eye at an entry of the eye that is at least 5 degrees from a perpendicular center line of the eye.

3. The method of claim 1, wherein the therapeutic agent is administered to the eye at an entry of the eye that is at least 20 degrees from a perpendicular center line of the eye.

4. The method of claim 1, wherein the therapeutic agent is administered to the eye at an entry of the eye that is at least 30 degrees from a perpendicular center line of the eye.

5. The method of any one of claim 1, wherein the eye is penetrated and the therapeutic agent is administered through a penetration site.

6. The method of claim 1, wherein the tangential administration of a composition comprising the therapeutic agent in a given time period results in pressure within the SCS that is reduced compared to the pressure within the SCS that results by perpendicular administration of a same volume of the therapeutic agent composition within a same time period.

7. The method of claim 6, wherein in any one of claims, tangential administration results in SCS pressure that is at least 5 percent less relative to the perpendicular administration.

8-9. (canceled)

10. The method of claim 1, further comprising:
penetrating the eye at a tangential angle with an insertion tip formed at a distal of a cannula through which the therapeutic agent is delivered into the SCS of the eye via a continuous and controlled infusion; and
delivering the therapeutic agent to the SCS of the eye via infusion tubing connected to the cannula.

11. The method of claim 10 further comprising deploying a sheath having a diameter greater than a diameter of the cannula and in which the cannula is housed.

12. The method of claim 1 wherein the cannula is bent at a particular angle to position the cannula on the eye.

13. The method of claim 1, wherein the insertion tip penetrates the SCS of the eye.

14. The method of claim 1, wherein the insertion tip is a needle.

15. The method of claim 1, wherein the tangential angle of the insertion tip is about 0-15 degrees.

16. The method of claim 1 wherein the therapeutic agent is administered via an insertion tip that is asymmetric.

17. The method of claim 16 wherein the insertion tip veers toward the sclera upon insertion to a subject's eye.

18. The method of claim 1, further comprising:
penetrating the eye at a tangential angle with an insertion tip formed at a distal of a cannula through which the therapeutic agent is delivered into the SCS the eye via a continuous and controlled infusion;
releasing the cannula into the eye; and
connecting infusion tubing to the cannula to deliver the therapeutic agent therethrough.

19. The method of claim 18 further comprising deploying a sheath having a diameter greater than a diameter of the cannula and in which the cannula is housed.

20. The method of claim 18 wherein the infusion tubing is connected to a controlled flow system to provide controlled flow rates of the therapeutic agent.

21-29. (canceled)

30. A method of delivering one or more therapeutic agents into an eye, comprising:
penetrating the eye at a tangential angle with an insertion tip formed at a distal of a cannula through which the one or more therapeutic agents are delivered into the eye via a continuous and controlled infusion; and
delivering the one or more therapeutic agents to the eye.

31-44. (canceled)

45. A surgical instrument for delivery of therapeutic agents into an eye, comprising:
a cannula connected to a handle of the surgical instrument and through which therapeutic agents are delivered into the eye via a continuous and controlled infusion; and
an insertion tip formed at a distal end of the cannula to penetrate tissue of the eye at a tangential angle.

46-79. (canceled)
